(19) United States
(12) Patent Application Publication
(10) Pub. No.: US 2003/0229517 A1
(43) Pub. Date: Dec. 11, 2003

(54) Medical management system and method

(76) Inventors: Peter Meserol, Montville, NJ (US); Jerry B. Slaven, Hillsborough, NJ (US); T. Jerome Madison, Skillman, NJ (US)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/386,271

(22) Filed: Mar. 11, 2003

Related U.S. Application Data
(60) Provisional application No. 60/364,710, filed on Mar. 15, 2002.

(51) Int. Cl. G06F 17/60; A61B 5/00

(52) U.S. Cl. 705/2; 705/7; 600/300

(57) ABSTRACT

A system and method is provided wherein managed care organizations (MCO) are invoiced directly by an entity which provides reports and/or data to the patient, to the patient's healthcare provider and to the patient's MCO, on a periodic basis. The reports are based on patient glucose or other disease monitoring tests inputted to a patient test analyzer monitor uniquely identified to the patient. The monitor is programmed to communicate over telephone lines or other communication system to the entity as frequently as nightly each day's tests with time and date stamps for each test. The entity optionally causes test units to be shipped directly to the patient as needed by monitoring patient supplies through the transmitted tests. Test results are correlated in reports to each patient and his or her healthcare provider and MCO. The reports provide timely information as to the status of the patient's control of the disease and results in timely provided therapy to the patient based on such current reporting, encouraging compliance by the patient. The MCO is billed periodically by the entity for the reports and all test units, if any, eliminating middlemen and improving administrative efficiency.

This invention relates to a medical management system for automatically monitoring patient medical condition and healthcare provider performance and for promoting prompt timely and immediate patient care in regard to chronic medical patient disease at a reasonable cost.

Diabetic patients test their blood glucose levels at home or at other non-clinical sites using a battery powered, hand-held analyzer and disposable test cells/strips. The patients place a drop of blood onto the test cell or strip and insert it into the analyzer which produces and displays glucose level data. The resultant data, displayed on an LCD contained in the analyzer, indicates the degree of blood glucose control achieved. The patient should use this information to alter his medication, exercise and/or food intake to maintain acceptable blood glucose control. However, this may or may not occur.

Blood glucose data is generally also captured and saved in the analyzer using an on-board memory that can be accessed on the analyzer or downloaded to a PC. This requires special software, along with appropriate cabling and a degree of computer skills. In practice, very few patients use the computer capture of data and even fewer healthcare providers have access to this clinically valuable data.

Healthcare providers, during their routine office visits with the patient, are especially interested in the degree of control the patient has achieved since their last office visit. While there are established testing protocols within the medical community, no record of patient testing compliance is currently readily available to the healthcare provider or relevant Managed Care Organization (MCO). MCOs are the principal organizations paying the costs for analyzers, test cells/strips and diabetes related healthcare costs. Nevertheless, current marketing efforts by manufacturers are virtually exclusively targeted to the patient. In this context, the patient purchases the equipment from the drug store, who then bills the MCO and/or causes the patient to pay. Eventually, then the patient will submit forms and receipts to the MCO for reimbursement.

When the patient goes to a healthcare provider, the healthcare provider may collect a blood sample and then submit the sample to a diagnostic lab. Results of the lab tests are normally not available to the healthcare provider while the patient is at the physician's office. The lab then bills the MCO. The visit to the healthcare provider is also billed to the MCO, either directly, or by paper work requesting reimbursement by the patient.

An article in Medical Technology Fundamentals, February 2001 by Daniel T. Lemaitre et al., Merrill Lynch, pages 259-283, discusses in detail the commercial present situation of diabetes testing and commercial market conditions for various companies in this field. At page 260, the article states that some companies ship test strips automatically to patients without determining the patient's need for supplies and submits to medicare for reimbursement. Insurance companies in many states are required to cover the cost of equipment, strips, monitors etc incurred by patients. Other aspects of this field are also discussed.

US patent publication 2001/0037060 Nov. 1, 2001 discloses a web site for glucose monitoring. The computer system uses glucose data downloaded from the patient's monitor. However, as noted above, many patients are either computer illiterate or do not want to use such systems. The data is provided back to the user or the user's care giver advising adjustment of food, exercise, medications and timed to stabilize glucose levels. Not all patients cooperate with such a system and not all patients even have PCs, especially the elderly and so on. Further, there is no way to insure the monitoring by the healthcare provider or MCO the use of such systems by the patient. These systems require incremental cost to the patient and/or the healthcare provider which further restricts acceptance and usage.

US publication no. 2001/0011224 discloses a modular self-care monitoring system which employs a compact microprocessor-based unit such as a video game system. It includes switches for controlling the device operation and a program cartridge. The cartridge adapts the microprocessor for use with a glucose or other health monitor. The system provides for transmission of signals to a remote clearing house or health care facility via telephone lines or other transmission media. Reports may be sent by facsimile to a health care professional. However, no incentive is provided to insure that the system is put into use and who is responsible for its cost. As a result, such a system is not in use in practice.

US publication no. 2001/0039372 discloses a health monitoring system for communication between at least one terminal device that moves with a person and a first center device. The terminal detects and diagnoses the health of the person and transmits the results to the first center device which stores the historical information and receives the result of the diagnosis and judges whether the detailed data is needed in accordance with the result of the diagnosis. A request command is issued of the detailed data to the terminal device when it judges that the detailed data is needed.

U.S. Pat. No. 6,032,137 discloses a remote image capture with centralized processing and storage. This system is related to credit card receipts and encrypts data, transmit the encrypted data to a central location and transforms the data to a usable form, verifies identification, using signatures and so on.

U.S. Pat. No. 4,731,726 discloses a monitor system which includes means for measuring blood glucose values and for generating glucose data signals. Monitor means is coupled to the measuring means and inputs patient data, means for transmitting and receiving data to and from the monitor means and computing means for receiving glucose data signal in connection with administration treatment for diabetes mellitus. This suffers the disadvantages of the other systems described above.

Health monitoring and reporting systems are also disclosed in U.S. Pat. Nos. 4,803,625; 5,307,263;5,549,117; 5,704,366;5,899,855; 6,134,504; 6,168,563; 6,295,506 and 6,290,646 and US Publication no. 2001/0004732.

U.S. Pat. No. 6,270,455 discloses a networked system for interactive communication and remote monitoring of drug delivery.

The problem with all of the above systems is that they do not promote use by the various entities that may use the system and impose incremental cost to the patient and/or healthcare provider and/or MCO. There is no incentive provided to the MCOs, the healthcare providers and the patients to use the systems. Some may require the patients to use computers which is not desirable as explained above. None discuss who is responsible for payment and installation of the system and which entity is responsible for operation of the system in the real world of MCOs, patients and healthcare providers, who are the primary people involved in the network. As discussed above and as shown in the discussion in the Merrill Lynch article cited above, none of these systems are discussed as being practical or being implemented by the primary entities involved in health care management and especially in connection with diabetes.

Diabetes is considered one of the highest cost diseases to treat. This is because the patients require constant monitoring, because if their disease goes out of control, the patient can contract serious complications, such as heart disease, blindness, loss of limbs or extremities, and other serious disabling expansion of the disease to its serious side effects, if not timely treated.

Timely monitoring of the patient is required to show out of control situations, and these out of control situations must be reported immediately to the health care professional, preferably the healthcare provider, as soon as possible. If a patient visits the healthcare provider's office and the healthcare provider does not have current information on the patient at the time of visit, the visit is of less significant value. Further, the MCO typically only receives bills from the various providers after the fact. The drug stores bill the MCOs for the equipment and test strips and test cells purchased as well as pharmaceuticals purchased. The healthcare provider also bills the MCO separately. Others involved in providing test strips and monitors to patients may also bill the MCO.

The MCO, however, does not have immediate knowledge of the current condition of the patient. To assist the patient in awareness of the disease, the MCO may send general disease related brochures to the patient with no assurances the patient may read the brochures or if read, take any concrete action based thereon. Further, because patient monitoring is self directed by the patient, the frequency and accuracy of the patient condition is subject to the patient reporting the data to his healthcare provider. Currently, there is no direct means for the MCO, healthcare provider and patient to all be involved in the specific condition of the patient and in ensuring the patient diligently monitors him or herself. While the prior art systems discussed above may alleviate the problem if properly implemented, currently there is no viable approach as to how to ensure such systems in practice are implemented in an industry wide program that is recognized as workable and affordable. As a result, as noted in the Merrill Lynch article, many systems are not presently properly used although they have been available for some time.

The present inventors recognize a need for an apparatus and method that provides incentive to the MCOs, the healthcare providers and the patients to not only implement an interactive monitoring system, but one that will motivate the patient to continue to use the system, that will not only not interfere with the practice of the healthcare provider, but will encourage communication to the healthcare provider, to the patient and to the MCO current status of the disease conditions of the patient and for the MCO to pay the involved costs in a practical manner. None of the prior art systems disclosed above resolve this need. A method for medical management reporting for optimizing patient cooperation with a healthcare provider and disease management programs implemented by MCOs and optimizing efficiency in payment for services and goods involved in the monitoring of the patient by the healthcare provider and managed care organization (MCO) in regard to a given disease according to the present invention comprises generating healthcare provider, patient and MCO reports and transmitting the reports to the healthcare provider, patient and MCO by an entity, each report manifesting the test analysis of human body fluids, most often blood, or interstitial fluid or other body fluids in regard to the current status of a specified disease in at least one patient at the time of each test periodically conducted by the at least one patient, each report including data manifesting the tested disease status for each test at the time of test, including the time and date stamp for each test and the patient being tested; periodically generating invoices and transmitting the invoices to the MCO by the entity, the invoices each manifesting an agreed to fee payable by the MCO to the entity for providing the reports to the healthcare provider, to the MCO and to the at least one patient on a periodic basis and for periodically supplying to the at least one patient by the entity any required test devices associated with the test conducted by the patient; and transmission by the MCO payments to the entity corresponding to the invoiced amounts for providing the reports, and the required test devices to the patient.

A medical management reporting system according to an aspect of the present invention for optimizing patient cooperation with a healthcare provider and relevant disease management programs and optimizing efficiency in payment for services and goods involved in the monitoring of the patient by the healthcare provider and managed care organization (MCO) in regard to a given disease comprises means for generating healthcare provider, patient and MCO reports to be provided by an entity, each report manifesting the test analysis of human body fluids in regard to the current status of a specified disease in at least one patient at the time of each test periodically conducted by the at least one patient and periodically communicated to the means for generating the reports, each report including data manifesting the tested disease status for each test at the time of test, including the time and date stamp for each test and the patient being tested; and invoice generating means coupled to the report generating means for generating periodic invoices to be provided to the MCO by the entity, the invoices each manifesting an agreed to MCO fee payable to the entity to provide the reports to the healthcare provider, MCO and at least one patient on a periodic basis and to periodically supply to the at least one patient any required test devices associated with the test conducted by the patient.

As a result, since the patient continuously receives a supply of test strips or cells, and since the patient receives periodic reports of his progress and the status of his disease, he or she is motivated to conduct the tests. The test data is readily communicated to the entity providing the reports by the patient test analyzer device which may include a modem and which the patient merely inserts into a mating cradle which, for example, provides a convenient connection to the telephone lines for automatic communication to the entity. This is a simple procedure for the patient who also is knowledgeable in self testing as in current test procedures conducted by the patient. The patient optimally inserts the test strip or cell in the analyzer device periodically as before. However, now the analyzed information is automatically communicated to the central processing center which generates the reports automatically and forwards the reports periodically to the patient, healthcare provider and MCO.

On the basis of these reports the MCO can monitor a number of patients simultaneously for each healthcare provider and can now also monitor each healthcare provider for performance. For example, the MCO can generate statistical reports on patient conditions of the different healthcare providers to see if more patients are out of control for a given healthcare provider than other healthcare providers. More importantly, the healthcare provider and MCO can both recognize out of control conditions in a timely manner and take immediate action to correct the condition before it transgresses into a more serious disease. There is thus provided motivation for all of the parties to participate and the MCO costs are reasonable considering the costs of present procedures. The patient is motivated by periodic reports showing his condition, the healthcare provider has the reports which he automatically receives and has to do nothing to obtain. More timely action can be taken by the healthcare provider at the time of the visit by the patient or immediate action can be taken by contacting the patient prior to a visit for those patients experiencing critical conditions. The MCO saves considerable paper work and associated costs and yet can provide more meaningful support to the patient as to the action necessary by the patient in view of the current reports.

In a further aspect, the means for generating the reports and the means for generating the invoices include a central processing unit (CPU) for operating the means for generating the reports and means for generating the invoices, further including a patient monitoring device unique to each of the at least one patient for the at least one patient use for receiving the at least one patient body fluid in one or more of the periodic tests entered by the at least one patient, the device including means for analyzing the received body fluid in regard to the current status of a specified disease and for producing and storing the data manifesting the analyzed disease status for each test, the device for generating the time and date stamp for each test, the device having a unique code corresponding to each the at least one patient; and communication means for communicating the unique code and the corresponding test data of each test inputted thereto and its corresponding time and date stamp to the CPU.

In a further aspect, the CPU includes means for corresponding the unique code to the corresponding at least one patient and test data.

In a further aspect, the communications means comprises a modem in the analyzer device for use in a mounting arrangement adapted to releasably receive and communicate the data and time and date stamp, the device including means to automatically periodically communicate the stored data and time and date stamp to the CPU at preprogrammed times.

In a further aspect, the device includes programmable means, the CPU being arranged to reprogram the programmable means to communicate to the CPU at a given schedule.

In a still further aspect, the device employs disposable test units for receiving the body fluids for analysis from the at least one patient, the CPU including means for tracking the number of test units available to the at least one patient for test and for causing replacement test units to be periodically shipped to the at least one patient prior to the depletion of test units then in the possession of the at least one patient without incremental cost to the patient.

In a further aspect, the disease is diabetes and the body fluids are blood, or any other bodily fluids manifesting glucose levels in the patient.

Preferably, in case of diabetes, the test unit is a test cell adapted for receiving patient blood.

Preferably, the generated report includes plotted charts and data for specific disease parameter values and the corresponding date of the test creating these values.

Preferably, the reports each include diabetes test data and corresponding date of test including HbA_{1c} values, glycated protein values, glucose summary data points, hypoglycemic values, and average glucose values over given time periods.

### BRIEF DESCRIPTION OF THE DRAWING

FIG. 1 is a schematic block diagram manifesting a system and method according to the present invention;

FIG. 2 is a diagrammatic representation of a patient analyzer and communication device for transmitting data via telephone lines;

FIG. 3 is a flow chart showing the use of the patient analyzer and test device;

FIG. 4 is a flow chart of the central entity process steps;

FIG. 5 is a flow diagrammatic representation of the generation of a healthcare provider report;

FIG. 6 is a flow diagrammatic representation of the generation of a managed care organization (MCO) report;

FIG. 7 is a flow diagrammatic representation of the generation of a patient report; and

FIG. 8 is an exemplary representative report for a diabetic person according to one aspect of the present invention.

### DEFINITIONS

The following terms as used herein are intended to interpreted in view of the following definitions in respect of the following detailed description and in the claims.

1. Managed Care Organization (MCO)—MCO means any organization which oversees the health care of patients and which is primarily responsible for paying the costs of such health care including HMOs (healthcare maintenance organizations), insurance companies, corporations which may or may not have internal health care management plans, medicare, medicaid, or any other entity responsible for payment of the costs of the health care of patients and directly or indirectly responsible for overseeing the health care of patients.

2. Healthcare Provider—The term healthcare provider includes any healthcare professional including physicians, physician assistants, nurses, nurse educators, nurse practitioners, nursing assistants, a hospital providing any of the aforementioned professionals, a clinic providing any of the aforementioned professionals, laboratories, diagnostic facilities and their personnel and so on responsible for the health care or for providing services related to the health care of a patient.

3. Report—As used herein, the term report includes any written or electronic form of data, whether or not displayed, either in raw data form or analyzed as to its significance including charts, graphs, plots, tables or summary information manifesting the significance of the data as applied to a given medical or medical related test.

4. Group of Patients—As used herein a group of patients is any number of patients receiving medical care from a healthcare provider and may include patients grouped by one or more of the patient's healthcare provider, geography, disease, economic status, level of disease control, costs or any other parameter which may provide statistical significant meaning to an MCO.

5. Patient—A person receiving the medical care of a health care provider.

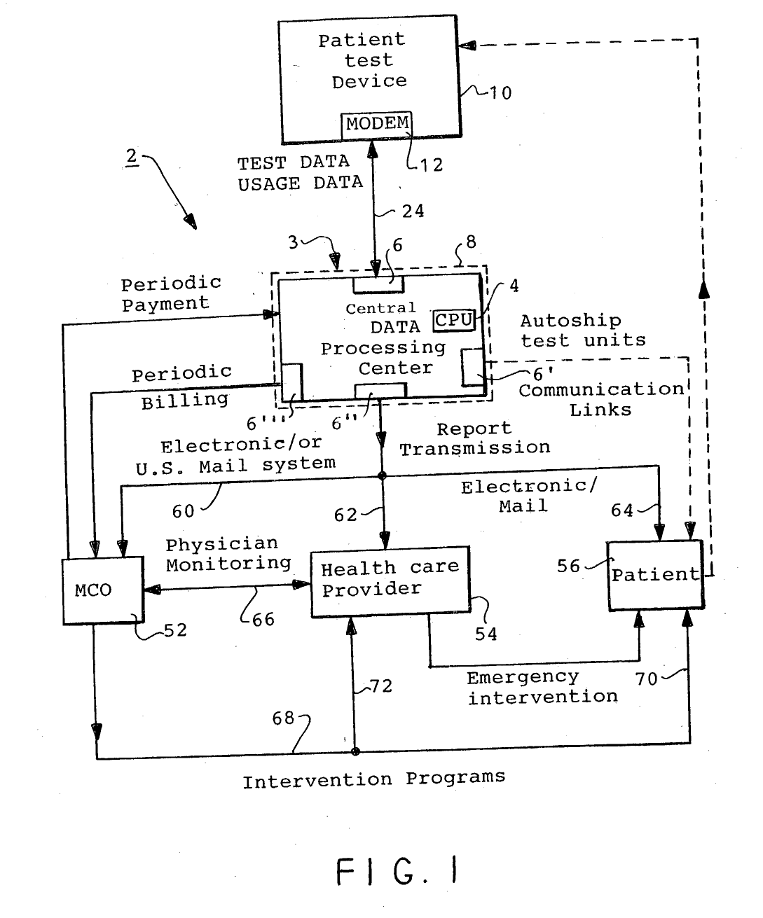

In FIG. 1, system 2 includes a host central data processing center 3, which includes a central processing unit 4, a computer (CPU), coupled to a communications links 6, 6′, 6″, and 6′″ such as telephone lines, dedicated communication lines, wireless systems or the internet and so on for communicating with outside entities or other devices of the system 2. The CPU is programmed to perform the desired functions to be explained in connection with FIGS. 3-8. The host center 3 is located in the facility of an entity 8 which operates the center 3 and which implements and pays for the cost of the center 3.

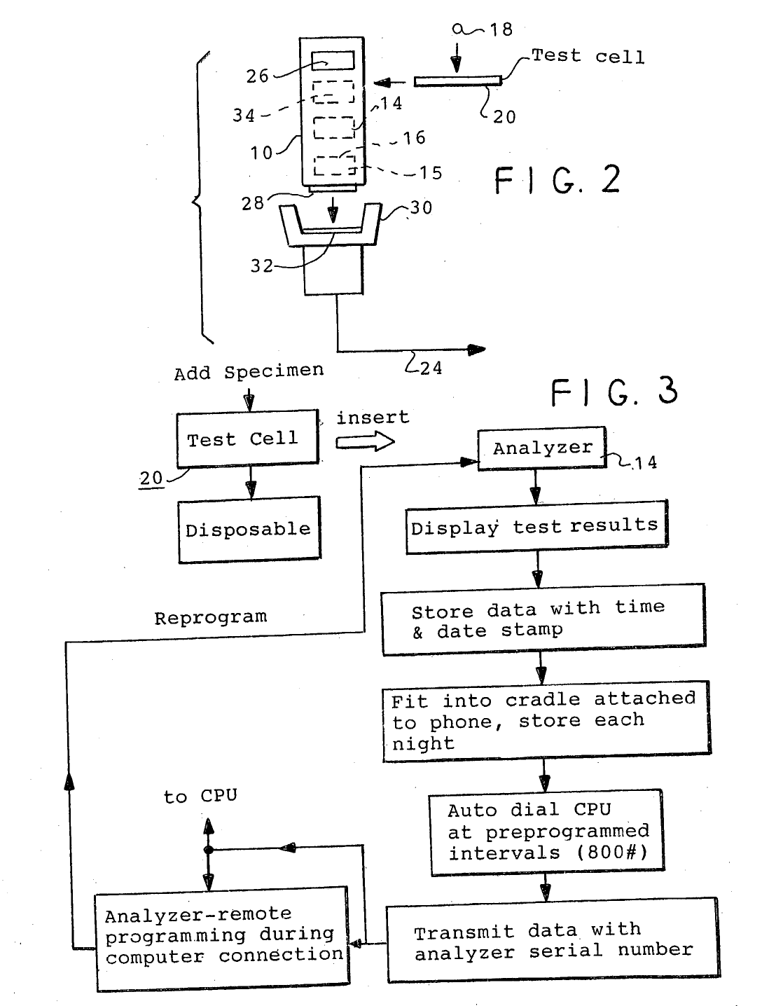

The center 3 receives data from the patient test device 10 via a modem 12 coupled to the patient test device 10 or included in the test device. The test device 10 may be a conventional analyzer for use in diabetes analysis, for example, or it may be a unique device developed for such analysis. Generally, such analyzers are commercially available as indicated in the various patents noted in the introductory portion. The test device 10, FIG. 2, for example, includes an analyzer 14 for producing data and for analyzing a blood drop 18 placed on a test cell 20. In the alternative, a conventional test strip may be used with a commercially available analyzer and test device. While diabetes is used as an example of the disease involved with system 2, it should be understood that the system 2 is useful for any disease in need of close monitoring with respect to the status of a patient's disease and/or costs of providing health care to the patient.

The patient places the test cell 20, FIG. 2, (or test strip as applicable) into the test device 10 which has a computer program 15 and related memory 16 for extracting data from the blood drop 18 and for storing the extracted data in the memory 16. The device 10 also has a unique code associated therewith and assigned to the patient permanently assigned to the device 10. This code may be a serial number or any other form of data uniquely associated with each patient. The program 15 is programmable via communicated program instructions communicated from the host computer CPU 4. The program 15 is programmed to dial up the CPU via the communication line 24, FIG. 1 and link 6. Line 24 may be land lines, wireless transmissions or manifest communication via the internet.

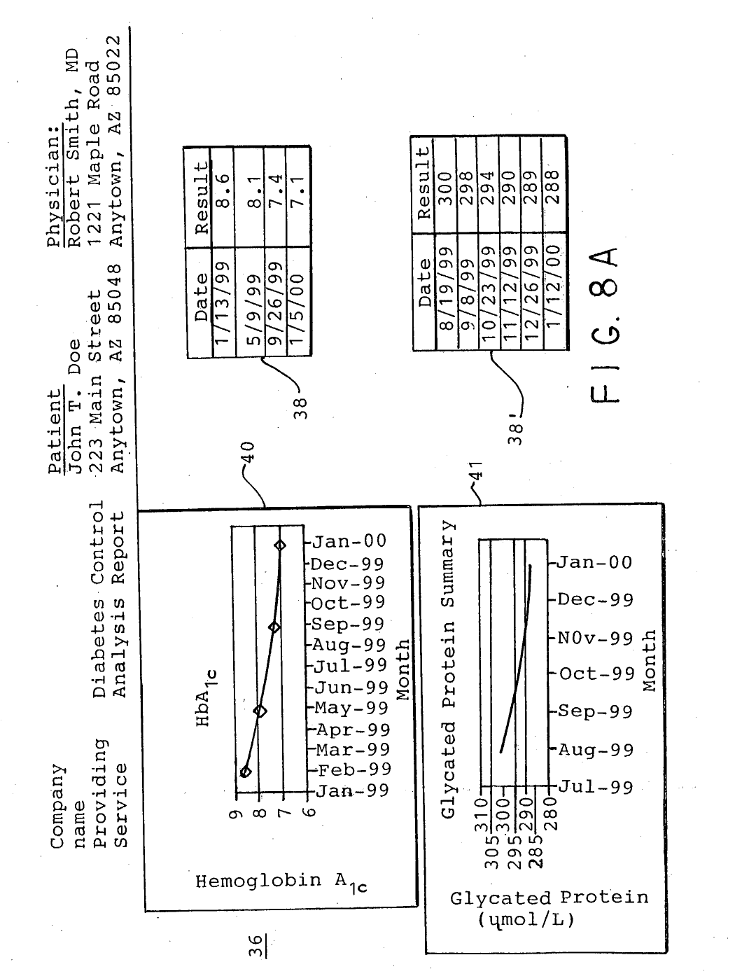

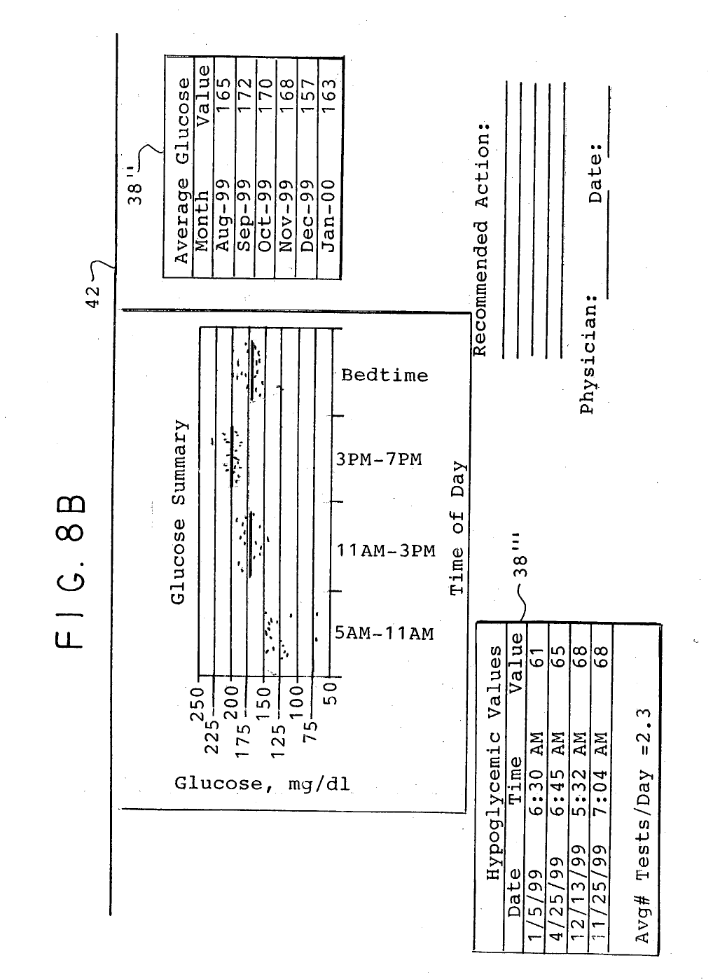

The device 10 has an LCD display 26 for displaying to the patient, using the device 10, the data produced in values such as shown in the report of FIG. 8 in terms of absolute values for each parameter or for selected parameters. The display may also indicate a warning for an out of control condition exhibited by any of the displayed parameters.

The device 10 has a connector 28 which mates with a connector 32 in cradle 30 which receives the device 10. The device 10 includes a modem 34 for communicating with the telephone line 24 or the modem may be in the cradle 30 as may be suitable for a given implementation. The instructions in the program 15 cause the modem to dial up the CPU periodically at predetermined times each month, preferably, for example, in the hours after 12 midnight. The patient is instructed to place the device 10 in its cradle 30 each night before retiring. The patient is also instructed to take periodic tests during each day or other prescribed routines. The dial up schedule for connecting the device 10 to the CPU 4 can be changed by the CPU to different hours or to communicate, if desired, more or less frequently, e.g., every week or every day. Thus the program 15 is arranged for two way communication with the CPU for transmitting test data and for receiving instructions for reprogramming the program 15. The program 15 has a code assigned thereto that is unique to that patient assigned the device 10. Each patient has his or her own assigned code.

The patient takes as many tests as prescribed in a given day or days and the data from the analysis of those tests is stored in memory 16 with the time and date of the test. Each test has its associated time and date stamp. The particular analyzed data produced and stored by device 10 is shown in the report of FIG. 8 by way of example for diabetes. Other diseases being monitored would have corresponding different data in its report.

In FIG. 3, the test cell 20 is disposable after one use. The test cells are optionally provided automatically by the entity 8, FIG. 1, which keeps track of the number of remaining test cells in the possession of the patient. Other arrangements may be used to supply the patient with test units if desired. The entity 8 supplies the patient with the initial quantity of test cells and replenishes the supply as it is used up by the patient in the preferred embodiment. The cells are automatically tracked by the CPU based on the imputed test data which in practice indicates the number of cells used each time the data is sent to the CPU. The CPU is programmed to analyze the data for test cell usage and this information is then transmitted as an order by the CPU to an order department in the entity 8. This order may be automatic and communicated directly to a manufacturer of the test cells who bills the entity 8 for the shipment of such cells to the patient(s).

It should be understood, that for other diseases or even in the case of diabetes, the patient may not necessarily be supplied test units in the system 2, which supplying function is optional. Such supplying of test units is preferable, but certain entities may desire other arrangements with respect to the supply of patients with test units, if at all required for a particular implementation.

In FIG. 3, the analyzer displays the test results for observation by the patient. It stores the data with the time and date stamp. It automatically dials the CPU at preprogrammed intervals and transmits the data to the CPU with the unique serial number of the analyzer which is associated with that particular patient.

In FIG. 1, the host center 3 generates the report of FIG. 8, by way of example, for a diabetic condition in the patient. The report 36 has the information as depicted in FIG. 8 which is self explanatory. This data is programmed to be converted by the CPU into chart form or other summary display for ease of observation by the recipient as evident from the charts 38, 38′, 38″ and 38′″ and graphs 40, 41 and 42. The charts 38 and so on indicate date and results of the various tests by the patient with respect to diabetes. The report lists the name of the entity 8, the healthcare provider and the patient names and addresses. Other reports (not shown) with different manifestations of the data or other data parameters such as patient, healthcare provider lists, geographic region of patients or healthcare providers, costs associated with the providers and/or patients and so on as may be desired by the MCO or healthcare provider are generated as needed or as specified for a given disease or need of the MCO.

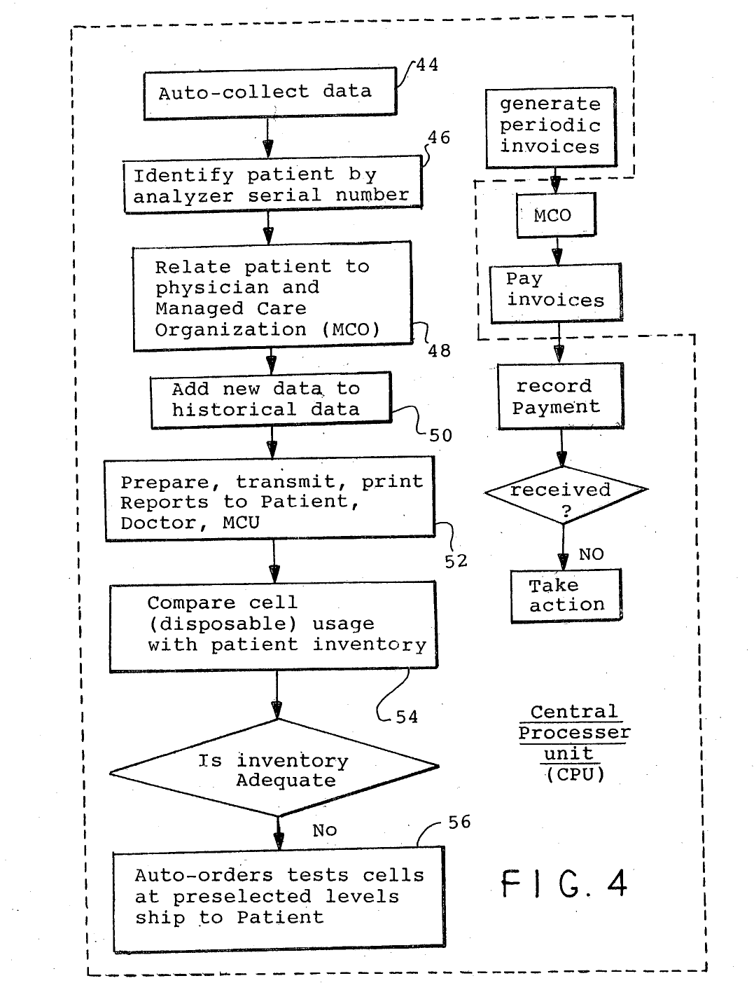

As shown in FIG. 4, the system 2, FIG. 1, automatically collects the data in step 44 and identifies the patient in step 46. In step 48, the patient is related to the healthcare provider and MCO. In step 50 new data is entered into the CPU memory. In step 52 the report 36 is generated at the entity 8 facility and transmitted to the patient, the healthcare provider and to the MCO at the same time. The report to the MCO and healthcare provider may be transmitted electronically or by mail. In the alternative, raw data may be provided the MCO if so desired. The patient report may be mailed by US Postal Service mail or by access to a secure personal website. The CPU then compares the patient usage of the test cells 20 with usage and patient inventory. If the inventory is depleted, automatic orders are generated for shipping replacement test cells directly to the patient according to predetermined quantities as associated with a given patient historical usage pattern.

In FIG. 1, the reports 36 are transmitted to the MCO, the healthcare provider 54 and patient 56 manifested by lines 60, 62 and 64 respectively. Line 66 represents healthcare provider monitoring by the MCO. For example, the MCO can enter the data from the reports 36 from the different healthcare providers in its operations with respect to distribution factors of out of control and in control patients for the disease being monitored. If one healthcare provider exhibits statistical abnormally high out of control patients, then the MCO can take action to discuss this with the healthcare provider The MCO can offer suggestions to the healthcare provider to bring its patients more into control with other healthcare providers in the MCO network by suggesting different protocols etc. in the treatment of the patient. The MCO also can direct specific action brochures and information directly to the patient, or enroll the patient in specific disease management intervention programs to assist the healthcare provider in correcting the out of control condition. Timely action by the patient to correct out of control conditions has a direct correlation in reducing the frequency and severity of serious disease complications. The patient, for example, may be directed to contact his or her healthcare provider immediately or even seek treatment in a hospital emergency room, if necessary. Thus the MCO can initiate intervention programs 68, FIG. 1, in a timely manner. Programs 68 can be directed at the patient at line 70 or healthcare provider at line 72.

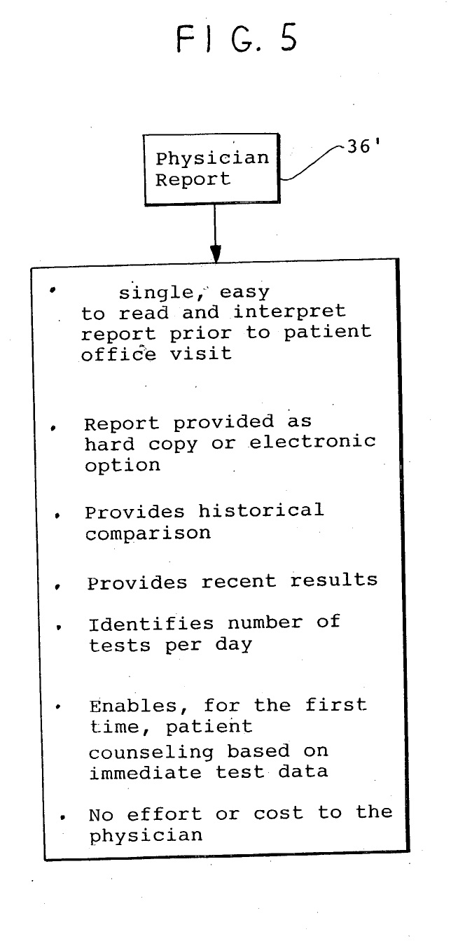

In FIG. 5, the healthcare provider report 36′ is received by the healthcare provider prior to the patient visit and thus the treatment is timely and more effective. The report may be hard copy or electronic. The report provides a historical comparison of data for each patient. It identifies the number of tests per day, and enables for the first time, patient counseling based on current test data at no effort or incremental cost to the patient or healthcare provider.

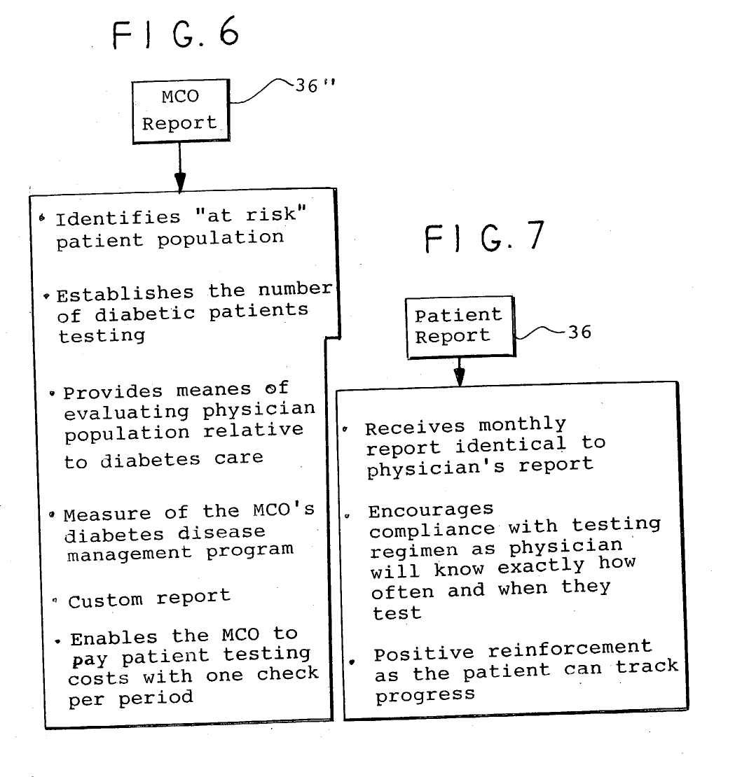

The report 36″ sent to the MCO, or any other third-party payor, FIG. 6, permits the MCO to identify “at risk” patient population and establishes the number of diabetic patients undergoing testing within that MCO patient population. It provides a means for evaluation of the healthcare provider population relative to the disease such as diabetes care, for example. It also provides the MCO a measure of its disease management program effectiveness. It can be a custom report unique to the MCO as requested by the MCO and modified into the CPU programming accordingly.

The patient report 36, FIG. 7, like the other reports, is generated and received periodically, monthly or at other frequencies according to the patients level of control or other factors warranting more frequent reports. Preferably reports are generated monthly. The patient report 36 may be identical to the healthcare provider report 36′ and encourages compliance with continuing the testing program.

Thus, the patient uses the blood glucose analyzer and test cell/strips as previously used in prior test systems. The test data is displayed on the analyzer for the patient's immediate action and the data is time and date stamped and stored in the on-board memory 16. This is essentially the same as the system employed by the patient previously.

Each night the patient places the analyzer device 10 in the cradle 30, similar in appearance to chargers used for cellular telephones. The cradle 30 is connected to a communication line 24 such as a telephone line. FIG. 2. This does not alter the normal use of the telephone.

The analyzer 14 is programmed to automatically “wake up” at specified and variable times, dial up the host entity CPU and automatically download all time and date stamped data along with the serial number of the analyzer to the CPU. The serial number is associated to the patient, the healthcare provider, and relevant MCO, and is automatically so associated by the CPU. During this connect time, the CPU is programmed as needed to communicate with the analyzer and down load such information such as changing the programmed dial up schedule for more or less frequent down load of data, leave alphanumeric messages such as “contact your healthcare provider” or provide early warnings relative to out of control conditions such as excessively high or low blood glucose readings. No patient name is associated with the data and the entity 8 provides privacy for the transmitted medical information. If necessary, this information could also be encrypted.

The entity 8 collects and analyzes the data at the CPU and prepares computerized reports for the patient and healthcare provider, highlighting the key clinically significant data including all or any of the following but not limited to daily glucose readings, bimonthly glycated protein levels and quarterly A_{1c} values. Consolidated reports are prepared and forwarded to the relevant MCO on a periodic basis.

A benefit of this system is the patient receives a monthly report, either hard copy or optionally, by accessing a corresponding secured website maintained by the entity 8 for that patient. This report informs the patient as to how well he or she is keeping the glucose levels under control, alerts the patient to the data that the healthcare provider will have in-hand for the next office visit, identifies the frequency of testing, and provides a reference to the historical glucose control, enabling the patient to note improvements or identify a developing negative trend. Knowing that the healthcare provider will have a record of the exact frequency and quantity of testing provides motivation to the patient to better comply with critical testing requirements and glucose control.

The system captures the number of tests consumed by the patient over a specified period and compares that number against the number of tests shipped directly to the patient by the entity 8. This enables the entity 8 to automatically refresh the patient's test supplies. No existing test system has this capability. This capability permits the entity 8 to automatically ship product and bill the relevant MCO automatically, for all of the services provided in one bill thereby removing the distribution middleman currently in use by blood glucose manufacturers and MCOs. This results in significant cost savings to the healthcare delivery system and to the MCO in direct costs as well as handling cost for multiple invoices each month.

The healthcare provider, preferably a physician, receives a single page hard copy report FIG. 8 prior to the patient office visit. Optional access to a secure website maintained by the entity 8 with current patient data may also be made available. This report enables the healthcare provider to quickly assess how well the patient is doing, how often the patient is testing, and identifies positive or negative trends. This critical information is not currently readily available to the healthcare provider, and in most cases, not accessible while the patient is in the healthcare providers office. The healthcare provider is able to devote the office visit time to modifying therapy and more effectively counsel the patient. Studies have shown that face to face counseling results in improved therapy compliance leading to reduced diabetic complications and reduced overall healthcare costs.

The ability to incorporate of the A_{1c} and glycated protein test results, a capability of an embodiment of the inventive system and method, further enhances the clinical status the healthcare provider is assessing. Since the healthcare provider receives this data prior to the patient visit, patients exhibiting dangerous glucose levels can be contacted immediately to rapidly address the critical situation. This capability does not exist with current glucose systems. The indicated average number of tests performed by the patient enables the healthcare provider to counsel the patient as necessary and promotes greater compliance with the testing protocols. No action is required of the healthcare provider to obtain this information. It is provided automatically on a periodic basis.

The MCO receives periodically, in this example, for example monthly or any other convenient period, electronically transmitted report (not shown) consisting of consolidated data custom analyzed per the specific MCOs needs or the same report received by the patient and healthcare provider as may be applicable. A separate report (not shown) identifies those patients within the MCO's plan that are “at risk,” that is, outside the acceptable parameters set by the MCO for its diabetic population. This enables the MCO to target disease management programs to the diabetic population in the greatest need, thereby reducing overall administrative costs. This more effectively utilizes disease management resources and reduces the cost impact of poor outcomes resulting from poor glucose control. This capability is not currently available to the MCO.

Additionally the MCO can monitor healthcare provider compliance with treatment protocols by comparing patient population data. Healthcare providers or healthcare provider groups, that are outside the acceptable standards can be encouraged to improve patient care both by assessing their performance compared to overall MCO healthcare provider population and by direct intervention by the MCO.

The ability to provide test cells directly to the patient population significantly reduces the administrative costs associated with tracking product through the pharmacy distribution chain. Patients that order more tests than are consumed can be readily identified and monitored, a capability not currently available. For example, periodic billing for consumables are consolidated and specific numbers captured to insure appropriate expense payments.

The group placement of patients for providing a third party payee for MCO diabetic subscribers of the MCO is novel and enables MCO control of costs and quality of services. That is, the system 2 permits the MCO to receive reports for all patients who are members of the MCO and who are patients of a given healthcare provider(s), and/or all patients with the same disease, and/or all patients or healthcare providers in a given geographic area and who are members of that MCO, and/or all invoices for common services and goods by cost and other statistical data useful to the MCO for determining out of control treatments or costs for different entities, providers or patient groups who are members of that MCO. This provides a revenue generating transaction between the MCO and the third party entity 8 not hereto before available in prior art systems, which resulted in lack of commercialization of such systems.

While diabetes is disclosed as the preferred embodiment, it is apparent that the disclosed invention is applicable to the control of various different kind of diseases which may exhibit similar type of control problems as diabetes. By providing a billing system that automatically covers the costs of patient conducted tests to the MCO from the entity providing the reporting service, and enabling the MCO to pay the entity directly for this service, all of the parties benefit without specific costs to each party. The MCO is willing to pay such fees due to significant cost savings afforded by the fact the patient is placed under greater control than provided by present systems and methodology. The MCO pays the report providing entity for both the reports and the test supplies and thus is more efficiently operated and minimizes costs to the MCO as incurred in present protocols. MCOs welcome such cost savings while at the same time enhancing disease control which provides even greater potential cost savings to the MCO.

It will occur to those of ordinary skill that various modifications may be made to the disclosed embodiments. The disclosed embodiments are given by way of illustration and not limitation. The scope of the invention is intended to be defined by the appended claims.

What is claimed is:

1. A medical management reporting system for optimizing patient cooperation with a healthcare provider and optimizing efficiency in payment for services and goods involved in the monitoring of the patient by the healthcare provider and managed care organization (MCO) in regard to a given disease comprising:
means for generating healthcare provider, patient and MCO reports to be provided by an entity, each report manifesting the test analysis of human body fluid in regard to the current status of a specified disease in at least one patient at the time of each test periodically conducted by the at least one patient and periodically communicated to the means for generating the reports, each report including data manifesting the tested disease status for each test at the time of test, including the time and date stamp for each test and the patient being tested; and
invoice generating means coupled to the report generating means for generating periodic invoices to be provided to the MCO by the entity, the invoices each manifesting an agreed to MCO fee payable to the entity to provide the reports to the healthcare provider, MCO and at least one patient on a periodic basis and to periodically supply to the at least one patient any required test devices associated with the test conducted by the patient.

2. The system of claim 1 wherein the means for generating the reports and the means for generating the invoices include a central processing unit (CPU) for operating the means for generating the reports and means for generating the invoices, further including a patient monitoring device unique to each of the at least one patient for the at least one patient use for receiving the at least one patient body fluids in one or more of the periodic tests entered by the at least one patient, the device including means for analyzing the received body fluids in regard to the current status of a specified disease and for producing and storing the data manifesting the analyzed disease status for each test, the device for generating the time and date stamp for each test, the device having a unique code corresponding to each the at least one patient; and
communication means for communicating the unique code and the corresponding test data of each test inputted thereto and its corresponding time and date stamp to the CPU.

3. The system of claim 2 wherein the CPU includes means for corresponding the unique code to the corresponding at least one patient and test data.

4. The system of claim 2 wherein the communications means comprises a modem adapted to communicate the device data and time and date stamp to the CPU, the device including means to automatically periodically communicate the stored data and time and date stamp to the CPU at preprogrammed times.

5. The system of claim 2 wherein the device includes programmable means, the CPU being arranged to reprogram the programmable means to communicate to the CPU at a given schedule.

6. The system of claim 2 wherein the device employs disposable test units for receiving the body fluids for analysis from the at least one patient, the CPU including means for tracking the number of test units available to the at least one patient for test and for causing replacement test units to be periodically shipped to the at least one patient prior to the depletion of test units then in the possession of the at least one patient without cost to the patient.

7. The system of claim 1 wherein the disease is diabetes and the body fluid are blood fluids or other bodily fluids manifesting glucose levels in the patient.

8. The system of claim 2 wherein the test unit is adapted for receiving patient blood or other bodily fluids.

9. The system of claim 1 wherein the generated report includes information for specific disease parameter values and the corresponding date of the test creating these values.

10. The system of claim 1 wherein the reports each include diabetes test data and corresponding date of test including one or more of HbA_{1c} values, glycated protein values, glucose summary data points, hypoglycemic values, and average glucose values over given time periods.

11. A medical management reporting system for optimizing patient cooperation with a healthcare provider and MCO, and for optimizing efficiency in payment for services and goods involved in the monitoring of the patient by a managed care organization (MCO) in regard to a given disease comprising:
a central processing unit (CPU);
a patient monitoring device for use by the patient, the device for receiving test units to which the patient applies body fluids for analysis by the device, the device including means for analyzing the applied body fluids for producing data manifesting the analysis and means for storing the analyzed data including time and date of the analysis, the body fluids manifesting the current status of a specified disease in the patient at the time of each test;
a test unit for use by the patient from a supply of test units in possession of the patient and to which test unit the patient applies his body fluids, the test unit for coupling to the device for performing the analysis;
communication means for communicating the stored data, time and date stamps of a plurality of tests to the CPU, the device including means for causing the communication means to communicate periodically with the CPU;
means responsive to the CPU for generating healthcare provider, patient and MCO reports and/or data to be provided by an entity, each report manifesting at least one of the test analysis and data on which the analysis is based of each test periodically communicated to the CPU by the communication means, each report including the information manifesting the tested disease status at the time of test; and
invoice generating means responsive to the CPU for generating periodic invoices to be provided to the MCO by the entity, the invoices each manifesting an agreed to fee payable to the entity which provides the reports to the healthcare provider, MCO and patient.

12. The system of claim 11 wherein the patient uses disposable test units for use with the monitoring device, the CPU for tracking the number of test units used by the patient and for ordering the test units for timely delivery to the patient for replacement of the depleted test units, the invoice agreed to fee including the cost of the patient supplied test units.

13. The system of claim 12 wherein the specified disease is diabetes.

14. The system of claim 13 wherein the reports each include diabetes test data and corresponding date of test including at least one of HbA_{1c} values, glycated protein values, glucose summary data points, hypoglycemic values, and average glucose values over given time periods.

15. The system of claim 11 wherein the device is assigned a unique code manifesting the associated patient, the CPU including means for corresponding the unique code and test data to the corresponding patient communicated thereto by the device and communication means.

16. A method of medical management reporting for optimizing patient cooperation with a healthcare provider and managed care organization (MCO), and for optimizing efficiency in payment for services and goods involved in the monitoring of the patient by the healthcare provider and MCO in regard to a given disease comprising:
motivating a patient to self test and generate data manifesting an analysis of his or her body fluids in regard to the current status of a specified disease in the patient at the time of each test periodically conducted by the patient;
motivating the patient to communicate the self test generated data to the entity in regard to the current status of a specified disease in a patient at the time of each test periodically conducted by the patient;
generating and transmitting to the MCO reports by an entity, each report manifesting at least one of the test data and analysis of human body fluids in regard to the current status of a specified disease in a patient at the time of each test periodically conducted by the patient, each report including data manifesting the tested disease status for each test at the time of test, including the time and date stamp for each test and information identifying the patient being tested;
generating periodic invoices and transmitting by the entity the generated invoices periodically to the MCO, the invoices each manifesting an agreed to fee payable to the entity for the entity to provide the reports to the MCO, healthcare provider and to the patient on a periodic basis;
ensuring by the entity the periodic supplying to the patient of any required test devices associated with the test conducted by the patient; and
paying the entity by the MCO on a periodic basis the agreed to fee for providing the reports and any supplied test devices in an agreed to period.

17. The method of claim 16 wherein the generating the reports and the generating the invoices include forming a central processing unit (CPU) for generating the reports and for generating the invoices, further including providing a patient monitoring device unique to the patient for receiving the at least one patient body fluid in one or more of the periodic tests entered by the patient, the device including means for analyzing the received body fluid in regard to the current status of a specified disease and for producing and storing the data manifesting the analyzed disease status for each test, the device for generating the time and date stamp for each test, the device having a unique code corresponding to each the at least one patient; and
communicating the unique code and the corresponding test data of each test inputted thereto and its corresponding time and date stamp periodically and automatically to the CPU.

18. The method of claim 16 wherein the generating the report includes generating a report manifesting the current status of a diabetic condition in the patient as periodically monitored by the patient.

19. A method for medical management reporting for optimizing patient cooperation with a healthcare provider and managed care organization (MCO) and optimizing efficiency in payment for services and goods involved in the monitoring of the patient by the healthcare provider in regard to a given disease comprising:
generating healthcare provider, patient and MCO reports and transmitting the reports to the healthcare professional, patient and MCO by an entity, each report manifesting the test analysis of human body fluids in regard to the current status of a specified disease in at least one patient at the time of each test periodically conducted by the at least one patient, each report including data manifesting the tested disease status for each test at the time of test, including the time and date stamp for each test and the patient being tested;
periodically generating invoices and transmitting the invoices to the MCO by the entity, the invoices each manifesting an agreed to fee payable by the MCO to the entity for providing the reports to the healthcare provider, to the MCO and to the at least one patient on a periodic basis and for optionally periodically supplying to the at least one patient by the entity any required test devices associated with the test conducted by the patient; and
transmission by the MCO payments to the entity corresponding to the invoiced amounts for providing the reports, and the required test devices to the patient.
